(19) United States
(12) Patent Application Publication
(10) Pub. No.: US 2025/0325208 A1
(43) Pub. Date: Oct. 23, 2025

(54) DEPRESSION MONITORING SYSTEM AND DEPRESSION EVALUATION METHOD

(71) Applicant: AI VALUE., Ltd., Taipei City (TW)

(72) Inventors: YU-SHU HUANG, Taoyuan (TW); CHIEN-YU TAO, New Taipei City (TW)

(73) Assignee: AI VALUE., Ltd., Taipei City (TW)

(21) Appl. No.: 19/176,321

(22) Filed: Apr. 11, 2025

Related U.S. Application Data
(60) Provisional application No. 63/635,643, filed on Apr. 18, 2024.

(51) Int. Cl. A61B 5/16; A61B 5/00; A61B 5/0205; A61B 5/024; A61B 5/11; A61B 5/384

(52) U.S. Cl. CPC A61B 5/165; A61B 5/0077; A61B 5/0205; A61B 5/024; A61B 5/1112; A61B 5/163; A61B 5/384; A61B 5/4803; A61B 5/681; A61B 5/6898; A61B 5/7267; A61B 5/7275; A61B 5/74

(57) ABSTRACT

A depression monitoring system is disclosed, equipped with a pre-trained depression evaluation model configured to implement a depression evaluation method. The method comprises a user data collection step, a depression evaluation step, and a warning step. In the user data collection step, the system collects a user's location data, movement range data, and physiological data. In the depression evaluation step, the collected data are input into the depression evaluation model, which generates a depression evaluation result. If the evaluation result indicates that the user is at risk of depression, the system transmits a notification to healthcare personnel associated with the user.

### TECHNICAL FIELD

The present invention relates to the field of mental health monitoring and, more particularly, to a system and method for evaluating and monitoring depression based on real-time behavioral and physiological data.

### BACKGROUND OF THE INVENTION

Depression is a prevalent mental disorder that affects a significant portion of the global population. Conventionally, the diagnosis of depression is determined based on the patient's score from standardized questionnaires, such as the Patient Health Questionnaire (PHQ), in combination with a psychiatrist's clinical judgment. However, clinical evidence suggests that the accuracy and reliability of such self-reported assessments may be compromised by various factors, including educational background, social desirability bias, and limited self-awareness.

To address these limitations, Taiwan Patent No. 1863817 discloses an electroencephalogram (EEG) analysis system designed to assist in evaluating depression. The system includes a frequency band filtering unit, a feature extraction unit, and a machine learning unit. The frequency band filtering unit processes the EEG signals from a subject to isolate specific sub-band signals. The feature extraction unit then extracts at least one feature from the sub-band signals, which is input into the machine learning unit to assess the subject's level of depression.

However, such EEG-based systems are typically installed in large medical institutions and are not suitable for continuous, daily monitoring of individuals who are at risk of developing depression or those already diagnosed. As such, these systems present significant limitations in practical application. In view of these drawbacks, the inventors of the present disclosure have devoted efforts to research and development and have accordingly developed an innovative depression monitoring system and depression evaluation method suitable for everyday use.

### SUMMARY OF THE INVENTION

The primary objective of the present invention is to provide a depression monitoring system equipped with a pre-trained depression evaluation model. The system is configured to collect, in a user's daily environment, location data, movement range data, and physiological data, and to evaluate these inputs via the depression evaluation model to generate a depression assessment result indicating whether the user is at high risk for depression.

If the evaluation result indicates that the user is at risk of depression, the system automatically transmits a notification to healthcare personnel associated with the user, thereby enabling timely care and intervention.

The disclosed depression monitoring system provides the following advantages: by collecting activity and physiological data (e.g., heart rate) in a non-invasive and continuous manner during everyday life, the system facilitates early detection of depressive symptoms. This innovation enables healthcare professionals to proactively identify high-risk individuals and administer appropriate treatment plans at earlier stages.

### BRIEF DESCRIPTION OF THE DRAWINGS

The technical characteristics of this disclosure will become apparent with the detailed description of preferred embodiments accompanied with the illustration of related drawings.

FIG. 1 is a first structural diagram of a depression monitoring system in accordance with an embodiment of the present invention.

FIG. 2 is a first block diagram of the depression monitoring system.

FIG. 3 is a second structural diagram of the depression monitoring system.

FIG. 4 is a second block diagram of the depression monitoring system.

FIG. 5 is a third structural diagram of the depression monitoring system.

FIG. 6 is a third block diagram of the depression monitoring system.

### DETAILED DESCRIPTION OF THE DISCLOSURE

### First Embodiment

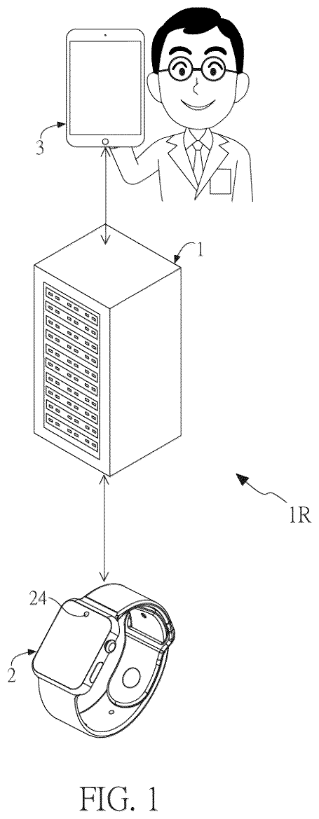

FIG. 1 is a first structural diagram of a depression monitoring system according to the present invention. As shown in FIG. 1, the depression monitoring system 1R of the present invention primarily comprises a master electronic device 1 and a first electronic device 2, and is configured to execute a depression evaluation method to assess whether a user is at high risk of suffering from depression. According to the design of the present invention, the depression evaluation method includes a user data collection step, a depression evaluation step, and a warning step.

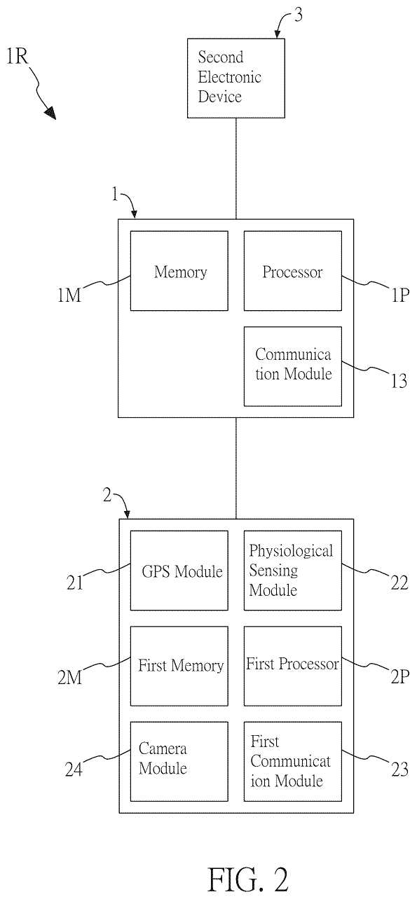

Further, FIG. 2 is a first block diagram of the depression monitoring system of the present invention. As shown in FIGS. 1 and 2, the master electronic device 1 may be, but is not limited to, an edge computing device or a cloud computing device, and includes a processor 1P, a memory 1M, and a communication module 13. On the other hand, the first electronic device 2 may be, but is not limited to, a smartwatch, a smartphone, a tablet, a notebook, or an embedded computer. For example, FIGS. 1 and 2 illustrate the first electronic device 2 as a smartwatch (or fitness tracker), held or worn by a user, and including a first processor 2P, a first memory 2M storing a first application program, a GPS module 21, a physiological sensing module 22, a first communication module 23, and a camera module 24. Additionally, in feasible embodiments, the memory 1M and the first memory 2M may each be, but are not limited to, a hard disk drive (HDD), a solid state drive (SSD), or a flash memory.

During normal operation, the depression monitoring system 1R first executes the user data collection step. At this point, the first processor 2P of the first electronic device 2 executes the first application program and is thereby configured to perform the following functions:
Collecting a location data and a movement range data of the user via the GPS module 21;
Measuring and collecting a physiological data of the user via the physiological sensing module 22; and
Transmitting the location data, the movement range data, and the physiological data to the master electronic device 1 via the first communication module 23.

It is worth noting that clinical studies indicate that users with depression tend to exhibit a rigid activity range (or social circle) and daily activity patterns with little noticeable variability. Therefore, in the user data collection step of the depression evaluation method of the present invention, the depression monitoring system 1R utilizes the user's smartwatch (i.e., the first electronic device 2) to collect the user's location data and movement range data in daily life, and analyzes the location data and movement range data to understand the user's activity range (or social circle) and daily activity patterns.

On the other hand, a clinical study by Goethe University in Germany found that, after monitoring the heart rates of 16 patients with major depressive disorder and 16 healthy volunteers over 4 days and 3 nights, the results showed that depressed patients had higher baseline heart rates and less fluctuation in heart rate over time. Accordingly, in the user data collection step of the depression evaluation method of the present invention, the depression monitoring system 1R utilizes the first electronic device 2 to collect the user's physiological data in daily life and analyzes the physiological data to understand changes in the user's physiological data. Specifically, the physiological data includes at least one of heart rate (HR), changes in heart rate over time, heart rate variability (HRV), and resting heart rate (RHR).

In greater detail, in the master electronic device 1, the memory 1M stores an application program and a pre-trained depression evaluation model, and the processor 1P executes the application program and is thereby configured to perform:
Receiving the location data, the movement range data, and the physiological data via the communication module 13; and
Inputting the location data, the movement range data, and the physiological data into the depression evaluation model, whereby the depression evaluation model outputs a depression evaluation result.

To reiterate, clinical studies indicate that, in addition to a rigid social circle (or activity range) and daily activity patterns with little noticeable variability, depressed patients exhibit relatively higher heart rates and smaller changes in heart rate over time. Therefore, in the depression evaluation step of the depression evaluation method of the present invention, the master electronic device 1 (i.e., an edge computing device or cloud computing device) is installed with a pre-trained depression evaluation model and is configured to input the location data, movement range data, and physiological data collected from the user by the first electronic device 2 into the depression evaluation model, whereby the depression evaluation model outputs a depression evaluation result.

Computer science (CS) engineers familiar with machine learning (ML) techniques will readily understand that the aforementioned depression evaluation model can be produced by performing machine learning training (or neural network training) on a classifier for depression evaluation using a training set. The training set includes first location data, first movement range data, and first physiological data collected from a plurality of individuals diagnosed with depression (i.e., training samples), as well as second location data, second movement range data, and second physiological data collected from healthy volunteers (i.e., gold standard samples).

Finally, in the warning step of the depression evaluation method of the present invention, if the depression evaluation result indicates that the user is at risk of depression, the master electronic device 1 immediately transmits a notification message to a second electronic device 3 via its communication module 13. As shown in FIGS. 1 and 2, the second electronic device 3 is owned by a healthcare personnel associated with the user and may be, but is not limited to, a smartwatch, a smartphone, a tablet, a notebook, a desktop computer, an all-in-one computer, or an embedded computer. In simple terms, if the depression evaluation result indicates that the user is at risk of depression, the depression monitoring system 1R of the present invention transmits a notification message to the healthcare personnel associated with the user, thereby providing immediate care and support to the user. This approach assists healthcare personnel in early detection of the user's depression and in providing appropriate treatment plans.

### Second Embodiment

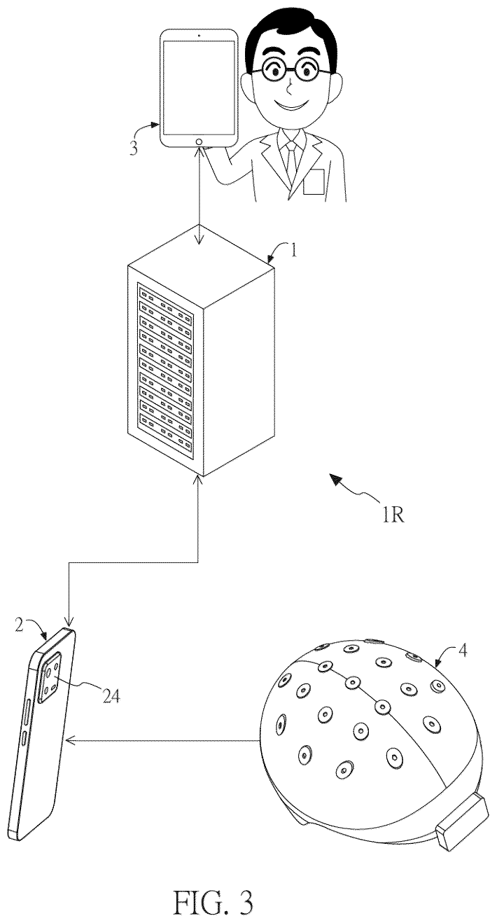

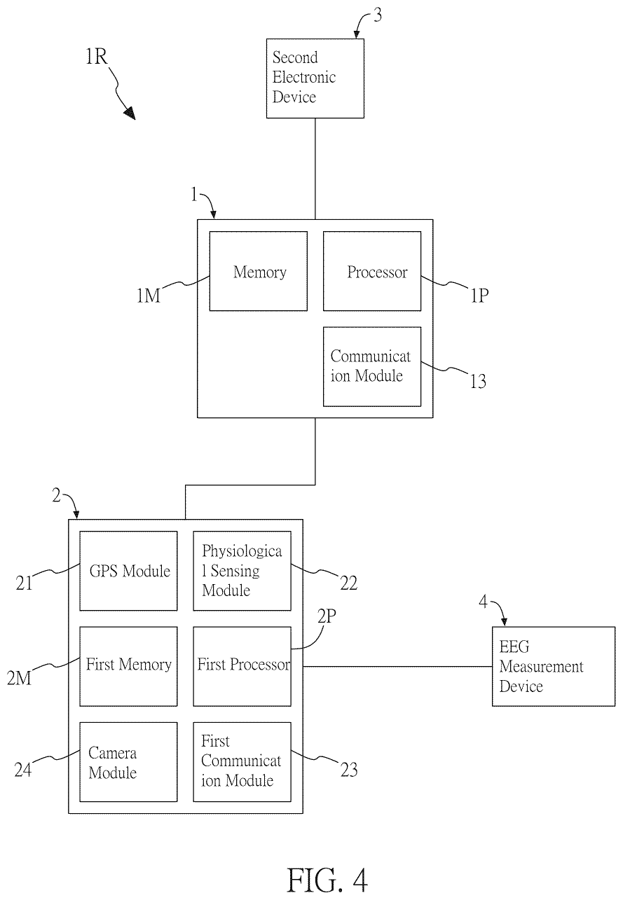

FIG. 3 is a second structural diagram of the depression monitoring system of the present invention, and FIG. 4 is a second block diagram of the depression monitoring system of the present invention. As shown in FIGS. 3 and 4, in the second embodiment, the depression monitoring system 1R of the present invention further comprises an EEG measurement device 4, which is communicatively linked to the master electronic device 1 or the first electronic device 2 and is configured to measure EEG data of the user. In feasible embodiments, the master electronic device 1 receives the EEG data directly from the EEG measurement device 4 or via the first electronic device 2. For example, FIGS. 3 and 4 illustrate the first electronic device 2 as a smartphone, held by a user, and the first electronic device 2 is communicatively connected to the EEG measurement device 4 to receive EEG data measured from the user. Subsequently, the first electronic device 2 transmits the user's location data, movement range data, and physiological data collected daily to the master electronic device 1, while also transmitting the EEG data, obtained periodically or non-periodically, to the master electronic device 1.

It should be additionally noted that when a smartphone is used as the first electronic device 2, its camera module 24 simultaneously serves as the physiological sensing module, and the first application program includes an iPPG or rPPG-based physiological parameter estimation algorithm. Ultimately, the processor 1P of the master electronic device 1 inputs the location data, the movement range data, the physiological data, and the EEG data into the depression evaluation model, whereby the depression evaluation model outputs the depression evaluation result. Furthermore, if the depression evaluation result indicates that the user is at risk of depression, the processor 1P transmits a notification message via the communication module 13 to healthcare personnel associated with the user, thereby providing immediate care and support to the user.

On the other hand, clinical studies also indicate that patients with depression frequently experience feelings of sadness, tearfulness, sorrow, irritability, fear, and anxiety. Additionally, clinical studies note that healthy individuals exhibit pupil dilation in response to incentives or rewards, whereas patients with depression show no similar response when faced with potential rewards. Therefore, in the second embodiment, the first electronic device 2 further includes a camera module 24, and the first processor 2P executes the application program and is thereby configured to control the camera module 24 to capture a user image of the user, and then transmit the user image to the master electronic device 1 via the first communication module 23.

It is worth noting that, in the second embodiment, the memory 1M further stores a pre-trained emotion recognition model, and the processor 1P executes the application program and is thereby configured to receive the user image via the communication module 13, and then input the user image into the emotion recognition model, whereby the emotion recognition model outputs facial emotion data. Ultimately, the processor 1P inputs the location data, the movement range data, the physiological data, and the facial emotion data and/or the EEG data into the depression evaluation model, whereby the depression evaluation model outputs the depression evaluation result. Specifically, the facial emotion data includes smile data and/or pupil change data.

### Third Embodiment

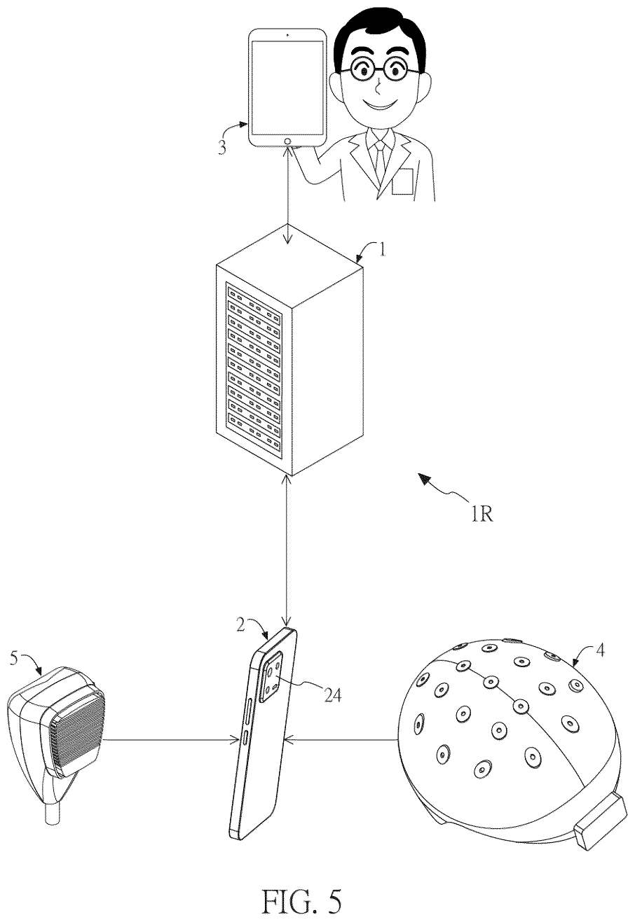

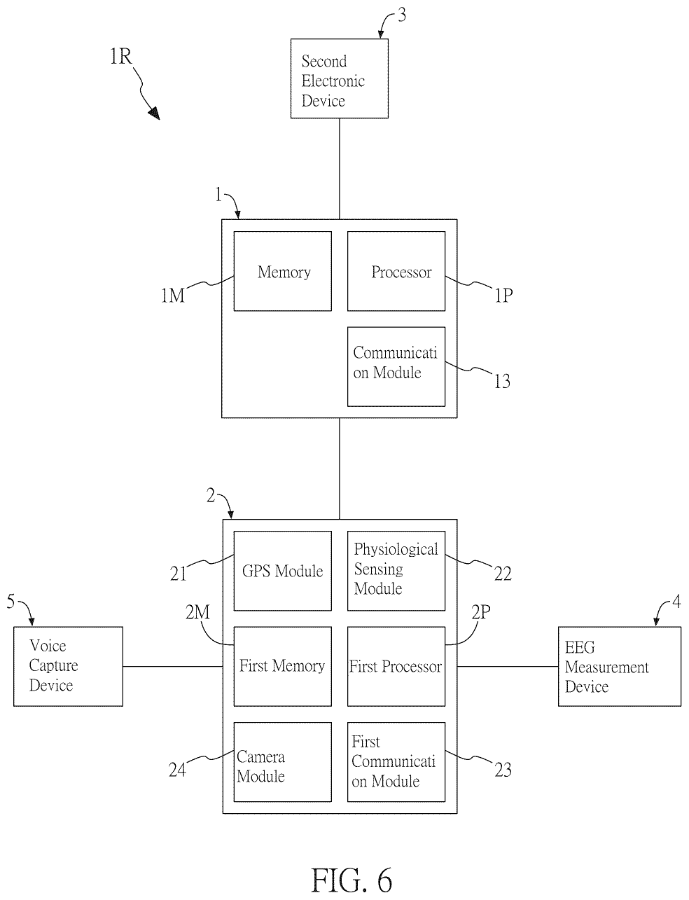

FIG. 5 is a third structural diagram of the depression monitoring system of the present invention, and FIG. 6 is a third block diagram of the depression monitoring system of the present invention. As shown in FIGS. 5 and 6, in the third embodiment, the depression monitoring system 1R of the present invention further comprises a voice capture device 5, which is communicatively linked to the master electronic device 1 or the first electronic device 2 and is configured to collect voice data from the user. In feasible embodiments, the master electronic device 1 receives the voice data directly from the voice capture device 5 or via the first electronic device 2. For example, FIGS. 5 and 6 illustrate the first electronic device 2 as a smartphone (or fitness tracker), held by a user, and the first electronic device 2 is communicatively connected to the voice capture device 5 to receive voice data collected from the user. Subsequently, the first electronic device 2 transmits the user's location data, movement range data, and physiological data collected daily to the master electronic device 1, while also transmitting the voice data, obtained periodically or non-periodically, to the master electronic device 1.

Clinical studies further indicate that as depression gradually worsens, the speech rate of patients typically slows down. Although patients may still be prone to irritability, they often lack the ability to express their emotions using complex language. Consequently, what is observed is a decline in the patients' expressive capacity-they hesitate to speak and appear very listless. Furthermore, when depression becomes significantly severe, the patients' speech rate may slow to the point of exhibiting “interruptions.” At times, after asking a question, it may take several minutes for the patient to respond. In some cases, patients may even lose the willingness to speak altogether.

Therefore, the processor 1P of the master electronic device 1 inputs the location data, the movement range data, the physiological data, and the voice data into the depression evaluation model, whereby the depression evaluation model outputs the depression evaluation result. Furthermore, if the depression evaluation result indicates that the user is at risk of depression, the processor 1P transmits a notification message via the communication module 13 to healthcare personnel associated with the user, thereby providing immediate care and support to the user.

What is claimed is:

1. A depression monitoring system, comprising:
a master electronic device, including a processor, a memory, and a communication module; and
a first electronic device, held or worn by a user, and including a first processor, a first memory storing a first application program, a GPS module, a physiological sensing module, and a first communication module;
wherein, the first processor executes the first application program and is thereby configured to perform:
collecting a location data and a movement range data of the user via the GPS module;
measuring and collecting a physiological data of the user via the physiological sensing module; and
transmitting the location data, the movement range data, and the physiological data to the master electronic device via the first communication module;
wherein, the memory stores an application program and a pre-trained depression evaluation model, and the processor executes the application program and is thereby configured to perform:
receiving the location data, the movement range data, and the physiological data via the communication module; and
inputting the location data, the movement range data, and the physiological data into the depression evaluation model, whereby the depression evaluation model outputs a depression evaluation result.

2. The depression monitoring system as described in claim 1, wherein the master electronic device is selected from the group consisting of an edge computing device and a cloud computing device.

3. The depression monitoring system as described in claim 1, wherein the physiological data includes at least one selected from the group consisting of heart rate (HR), changes in heart rate over time, heart rate variability (HRV), and resting heart rate (RHR).

4. The depression monitoring system as described in claim 1, wherein the first electronic device is selected from the group consisting of a smartwatch, a smartphone, a tablet, a notebook, and an embedded computer.

5. The depression monitoring system as described in claim 1, wherein the memory and the first memory are each selected from the group consisting of a hard disk drive (HDD), a solid state drive (SSD), and a flash memory.

6. The depression monitoring system as described in claim 1, wherein the depression evaluation model is produced by performing machine learning training on a classifier for depression evaluation using a training set, and the training set includes: first location data, first movement range data, and first physiological data collected from a plurality of individuals diagnosed with depression, as well as second location data, second movement range data, and second physiological data collected from healthy individuals.

7. The depression monitoring system as described in claim 1, further comprising:
an EEG measurement device, communicatively linked to the master electronic device or the first electronic device, and configured to measure EEG data of the user;
wherein the master electronic device receives the EEG data directly from the EEG measurement device or via the first electronic device.

8. The depression monitoring system as described in claim 7, wherein the processor executes the application program and is thereby configured to perform:
inputting the location data, the movement range data, the physiological data, and the EEG data into the depression evaluation model, whereby the depression evaluation model outputs the depression evaluation result.

9. The depression monitoring system as described in claim 7, wherein the first electronic device further includes a camera module, and the first processor executes the application program and is thereby configured to perform:
controlling the camera module to capture a user image of the user; and
transmitting the user image to the master electronic device via the first communication module.

10. The depression monitoring system as described in claim 9, wherein the memory further stores a pre-trained emotion recognition model, and the processor executes the application program and is thereby configured to perform:
receiving the user image via the communication module;
inputting the user image into the emotion recognition model, whereby the emotion recognition model outputs facial emotion data; and
inputting the location data, the movement range data, the physiological data, and the facial emotion data and/or the EEG data into the depression evaluation model, whereby the depression evaluation model outputs the depression evaluation result;
wherein the facial emotion data includes smile data and/or pupil change data.

11. The depression monitoring system as described in claim 10, further comprising:
a voice capture device, communicatively linked to the master electronic device or the first electronic device, and configured to collect voice data from the user;
wherein the master electronic device receives the voice data directly from the voice capture device or via the first electronic device.

12. The depression monitoring system as described in claim 11, wherein the processor executes the application program and is thereby configured to perform:
inputting the location data, the movement range data, the physiological data, and the facial emotion data, the EEG data, and/or the voice data into the depression evaluation model, whereby the depression evaluation model outputs the depression evaluation result.

13. The depression monitoring system as described in claim 12, wherein the processor executes the application program and is thereby configured to perform:
in the case where the depression evaluation result indicates that the user is at risk of depression, transmitting a notification message to a second electronic device via the communication module;
wherein the second electronic device is owned by a healthcare personnel and is selected from the group consisting of a smartwatch, a smartphone, a tablet, a notebook, a desktop computer, an all-in-one computer, and an embedded computer.

14. A depression evaluation method, executed by a system configured to perform depression monitoring, wherein the system is installed with a pre-trained depression evaluation model; the depression evaluation method comprising:
user data collection step: collecting a location data, a movement range data, and a physiological data of a user; and
depression evaluation step: inputting the location data, the movement range data, and the physiological data into the depression evaluation model, whereby the depression evaluation model outputs a depression evaluation result.

15. The depression evaluation method as described in claim 14, wherein the physiological data includes at least one selected from the group consisting of heart rate (HR), changes in heart rate over time, heart rate variability (HRV), and resting heart rate (RHR).

16. The depression evaluation method as described in claim 14, wherein the depression evaluation model is produced by performing machine learning training on a classifier for depression evaluation using a training set, and the training set includes: first location data, first movement range data, and first physiological data collected from a plurality of individuals diagnosed with depression, as well as second location data, second movement range data, and second physiological data collected from healthy individuals.

17. The depression evaluation method as described in claim 14, wherein the system, during the user data collection step, simultaneously collects EEG data of the user.

18. The depression evaluation method as described in claim 15, wherein the system, during the depression evaluation step, simultaneously inputs the EEG data into the depression evaluation model, causing the depression evaluation model to output the depression evaluation result based on the location data, the movement range data, the physiological data, and the EEG data.

19. The depression evaluation method as described in claim 15, wherein the system, during the user data collection step, simultaneously collects facial emotion data of the user.

20. The depression evaluation method as described in claim 17, wherein the system, during the depression evaluation step, simultaneously inputs the EEG data and/or the facial emotion data into the depression evaluation model, causing the depression evaluation model to output the depression evaluation result based on the location data, the movement range data, the physiological data, and the facial emotion data and/or the EEG data.
